(19) United States
(12) Patent Application Publication
(10) Pub. No.: US 2009/0092689 A1
(43) Pub. Date: Apr. 9, 2009

(54) COMPOSITION AND METHOD FOR SUPPORTING FEMALE SEXUAL HEALTH

(75) Inventor: Barry Jay Heck, Marina del Rey, CA (US)

Correspondence Address: SYLMARK, INC., 7821 Orion Avenue, SUITE 200, Lake Balboa, CA, 91406, US

(73) Assignee: Sylmark Holdings Limited, Dublin (IE)

(21) Appl. No.: 12/241,421

(22) Filed: Sep. 30, 2008

Related U.S. Application Data
(60) Provisional application No. 60/997,749, filed on Oct. 5, 2007.

(51) Int. Cl. A61K 36/254; A61P 15/00

(52) U.S. Cl. 424/728

(57) ABSTRACT

The present invention relates generally to a composition and method for supporting female sexual wellness by providing a nutritional supplement comprising of acanthopanax gracilistylus, glycyrrhiza glabra, chrysanthemum morbifolium, corydalis ambigua, and ligusticum in combination, and more preferably, comprising of acanthopanax gracilistylus, chrysanthemum morbifolium, and ligusticum in combination. The invention synergistically supports female sexual health when taken regularly as a dietary supplement.

### RELATED APPLICATION

This application claims priority from Provisional Application No. 60/997,749 file Oct. 5, 2007.

### FIELD OF INVENTION

The present invention relates generally to a composition and method for supporting female sexual wellness by providing a nutritional supplement comprising of acanthopanax gracilistylus, glycyrrhiza glabra, chrysanthemum morbifolium, corydalis ambigua, and ligusticum in combination, and more preferably, comprising of acanthopanax gracilistylus, chrysanthemum morbifolium, and ligusticum in combination. The invention synergistically supports female sexual health when taken regularly as a dietary supplement.

### BACKGROUND OF THE INVENTION

Sex may be a natural act, but for millions of women who suffer from sexual dysfunction, it can be vexingly unattainable. Female sexual dysfunction, decreased libido, arousal and orgasmic disorder are significant problems that affect approximately forty million women in the United States. The etiology of these troubles can be based on a number of psychological, anatomical, emotional, physiological, circulatory and other conditions.

Alleviating sexually related problems not only enhances sexual pleasure but can serve to manage stress, strengthen interpersonal relationships, maintain a positive mental outlook, and generally enable one to feel good and enjoy life. It is therefore desirable to provide a composition that would support and improve female sexual health.

### DETAILED DESCRIPTION OF PREFERRED EMBODIMENTS

The present invention is directed to a composition and method for supporting female sexual wellness. In a preferred embodiment of the invention, a nutritional supplement is provided comprising acanthopanax gracilistylus, glycyrrhiza glabra, chrysanthemum morbifolium, corydalis ambigua, and ligusticum in combination, and more preferably, comprising acanthopanax gracilistylus, chrysanthemum morbifolium, and ligusticum in combination. The invention synergistically supports female sexual health when taken regularly as a dietary supplement.

The composition of the present invention is a precursor to nitric oxide and promotes vascular circulation by increasing vascular flow and increasing vascular dilation. In a preferred embodiment, the composition is administered as a dietary supplement in the form of a capsule, tablet, powder or as an aqueous composition. If administered as a capsule, tablet, or powder, an amount of each of the constituents is mixed in accordance with standard pharmaceutical practice to form a unit dosage that can be swallowed by taking water with the unit dosage. The size of the tablet or capsule will typically be less than about a gram to maximize the ease of swallowing.

In a preferred embodiment of the present invention, the composition is administered in the form of a 500 mg capsule. Table I sets forth the preferred amounts in which the constituents are combined to form a unit dosage of 500 mg.

TABLE I

| Constituent | Preferred Amount |
|---|---|
| Acanthopanax gracilistylus | 166 mg |
| Ligusticum Wallichii | 166 mg |
| Chrysanthemum morbifolium | 168 mg |

The capsule is preferably made of 100% pure gelatin in the size 00. In general, the administration of the composition of this invention is carried out on a daily basis for a period of time sufficient to improve sexual health. The time and amount administered may vary from subject to subject. In a preferred embodiment of the invention, the recommended daily dosage is one capsule per day, taken between 4 p.m. and 6 p.m. The capsules are preferably taken regularly as a daily supplement. For effective results, the tablets should be taken regularly for no less than four consecutive days.

Table II sets forth the preferred ingredients, source, concentrations and percentages by volume of a preferred embodiment of the inventive composition.

TABLE II

| | | | % by |
|---|---|---|---|
| Ingredients | Source | Concentration | Volume |
| Acanthopanax (spiny panax) | Root's bark | 100% | 20.0% |
| (acanthopanax gracilistylus) | | | |
| Licorice | Entire root | 100% | 20.0% |
| (glycyrrhiza glabra) | | | |
| Chrysanthemum | Flower | 100% | 20.0% |
| (Chrysanthemum morbifolium) | | | |
| Corydalis | Entire root | 100% | 20.0% |
| Ligusticum | Entire root | 100% | 20.0% |

In one embodiment of the invention, the composition of the present invention is a part of a more comprehensive dietary supplement, containing up to 100% of the recommended daily values for certain vitamins and minerals. For example, in addition to the preferred ingredients described above (namely, acanthopanax gracilistylus, glycyrrhiza glabra, chrysanthemum morbifolium, corydalis, and ligusticum), the composition may include one or more of the following: Vitamins A, C and E; Vitamin B-complex, including thiamin, riboflavin, niacin, vitamin B-6, folate, vitamin B-12, biotin, pantothenic acid, zinc, calcium, iron and selenium.

The present invention includes a method of supporting female sexual health by administering the inventive composition to female humans. The regular administration of the composition over time results in improved sexual health in women. In a double-blind placebo controlled clinical study, the effectiveness of the inventive composition was tested as follows. Forty-eight participants were selected. The patients included those with sexual function complaints as well as those wishing to enhance their sexual pleasure. The sexual health of the participants was determined through an assessment questionnaire derived from and based upon the FSFI, Female Sexual Function Index.

During a two week clinical trial, some of the subject were given a placebo capsule and some of the subjects were given a capsule containing an embodiment of the present invention. Specifically, the non-placebo capsule contained 500 mg comprising equal ratios of acanthopanax gracilistylus, chrysanthemum morbifolium, and ligusticum. The placebo capsules contained 500 mg of brown glucose. The appearance of the placebo and non-placebo capsules was identical. The participants each took one capsule daily for two weeks. After the two week clinical trial, the sexual health questionnaire was re-administered.

The questionnaire focused on seven (7) areas:
1. sexual desire
2. satisfaction with sex life
3. frequency of sexual relations with partner
4. frequency of orgasms
5. frequency of multiple orgasms
6. clitoral sensation to stimulation
7. vaginal lubrication

The study found that the inventive formulation produced statistically significant improvement in vaginal lubrication, sexual desire, clitoral sensation, orgasmic potential and satisfaction. Some of the results are summarized in Table III below:

TABLE III

| Sexual Health | Total Reported | Placebo % |
|---|---|---|
| Questions | % Improvement | Improvement |
| Vaginal lubrication | 73.9% | 15.1% |
| Sexual desire | 63.3% | 16.7% |
| Clitoral sensation | 75.5% | 15.7% |
| to stimulation | | |
| Satisfaction with | 72.9% | 13.1% |
| sex life | | |
| Ease of achieving | 64.4% | 21.8% |
| orgasm | | |
| Ease of achieving | 45.2% | 9.6% |
| multiple orgasms | | |
| Frequency of sexual | 60.9% | 10.1% |
| relations with | | |
| partner | | |

The positive outcome of the clinical studies was a result of the formulation of the present invention stimulating the medulla oblongata and increasing blood flow to the genitals. The combination of acanthopanax gracilistylus, chrysanthemum morbifolium, and ligusticum synergistically acts as a precursor to Nitric Oxide (NO), increases NO production and facilitates and increases microvascular circulation by acting as a vasodilator. As a result, subjects experience increased sexual arousal, increased clitoral engorgement and erection, increased vaginal lubrication and increased ease of achieving orgasms. Furthermore, the composition acts as a mood enhancer, promoting a relaxed state of mind, relaxing the muscles, and regulating mood and emotions.

In a preferred embodiment of the invention, the composition comprises all herbal ingredients and more preferably, natural (non-manufactured) herbal ingredients. The natural herbal ingredients ensure that the consumer does not experience any negative side effects resulting from the consumption of the supplement.

Many modifications and other embodiments of the invention set forth herein will come to mind to one skilled in the art to which the invention pertains having the benefit of the teachings presented in the foregoing descriptions and the associated tables. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A composition for supporting female sexual health comprising acanthopanax gracilistylus, chrysanthemum morbifolium, and ligusticum.

2. The composition of claim 1 wherein acanthopanax gracilistylus, chrysanthemum morbifolium, and ligusticum are provided in substantially equal ratios.

3. The composition of claim 1 wherein said composition is a dietary supplement.

4. The composition of claim 1 wherein said composition is a capsule.

5. The composition of claim 1, further comprising glycyrrhiza glabra and corydalis ambigua.

6. The composition of claim 5 wherein acanthopanax gracilistylus, chrysanthemum morbifolium, ligusticum, glycyrrhiza glabra and corydalis ambigua are provided in substantially equal percentages by volume.

7. A method of supporting female sexual health comprising administering to a female human a composition comprising acanthopanax gracilistylus, chrysanthemum morbifolium, and ligusticum.

8. The method of claim 7 wherein acanthopanax gracilistylus, chrysanthemum morbifolium, and ligusticum are provided in substantially equal ratios.

9. The method of claim 7 wherein said composition is a dietary supplement.

10. The composition of claim 7 wherein said composition is a capsule.

11. The composition of claim 7, further comprising glycyrrhiza glabra and corydalis ambigua.

12. The composition of claim 11 wherein acanthopanax gracilistylus, chrysanthemum morbifolium, ligusticum, glycyrrhiza glabra and corydalis ambigua are provided in substantially equal percentages by volume.
